SEQUENCE LISTING (14 sequences)

SEQ ID NO: 1 - protein, 5 aa, Artificial Sequence
  Synthetic peptide
XXGXX

SEQ ID NO: 2 - protein, 6 aa, Artificial Sequence
  Synthetic peptide
XXHXXW

SEQ ID NO: 3 - protein, 5 aa, Artificial Sequence
  Synthetic peptide
XKGHK

SEQ ID NO: 4 - protein, 5 aa, Artificial Sequence
  Synthetic peptide
XKGHK

SEQ ID NO: 5 - protein, 4 aa, Artificial Sequence
  Synthetic peptide
XGHK

SEQ ID NO: 6 - protein, 4 aa, Artificial Sequence
  Synthetic peptide
XGHK

SEQ ID NO: 7 - protein, 5 aa, Artificial Sequence
  Synthetic peptide
XKGHK

SEQ ID NO: 8 - protein, 4 aa, Artificial Sequence
  Synthetic peptide
XGHK

SEQ ID NO: 9 - protein, 5 aa, Artificial Sequence
  Synthetic peptide
XHXRW

SEQ ID NO: 10 - protein, 6 aa, Artificial Sequence
  Synthetic peptide
XKHXRW

SEQ ID NO: 11 - protein, 5 aa, Artificial Sequence
  Synthetic peptide
XHWRW

SEQ ID NO: 12 - protein, 6 aa, Artificial Sequence
  Synthetic peptide
XKHWRW

SEQ ID NO: 13 - protein, 5 aa, Artificial Sequence
  Synthetic peptide
XHXXW

SEQ ID NO: 14 - protein, 4 aa, Homo sapiens
GSSG

(19) United States
(12) Patent Application Publication
(10) Pub. No.: US 2010/0310484 A1
(43) Pub. Date: Dec. 9, 2010

(54) NOVEL PEPTIDES, USE THEREOF IN COSMETIC AND COSMECEUTIC APPLICATIONS, AND COMPOSITIONS COMPRISING SAME

(75) Inventors: Michel Hocquaux, Paris (FR); Estelle Loing, Quebec (CA); Philippe Bedos, Donneville (FR)

Correspondence Address: SCHWEGMAN, LUNDBERG & WOESSNER, P.A., P.O. BOX 2938, MINNEAPOLIS, MN, 55402, US

(73) Assignee: Innovactiv Inc., Rimouski (CA)

(21) Appl. No.: 12/452,381

(22) PCT Filed: Jun. 30, 2008
(86) PCT No.: PCT/CA2008/001227

§ 371 Date: Aug. 25, 2010

Related U.S. Application Data
(60) Provisional application No. 60/947,148, filed on Jun. 29, 2007.

(51) Int. Cl. A61K 8/64; C07K 5/11; C07K 5/083; C07K 7/06; C07K 5/117; A61K 38/07; A61K 38/06; A61Q 17/04; A61Q 19/08; A61P 17/02; C12N 5/07

(52) U.S. Cl. 424/60; 530/330; 530/331; 514/21.9; 435/375

(57) ABSTRACT

A peptide of formula I (SEQ ID NO: 1): Lip-A-Gly-His-B-R (I) wherein: Lip is a lipoyl residue of R or S configuration; A is absent or is a lysine residue of configuration L or D; GIy is a glycine residue; His is a histidine residue of configuration L or D; B is a lysine residue of configuration L or D, or a lysine residue of configuration L or D in which the NH2 group of the side chain comprises a modification, wherein said modification is (i) a replacement with a hydrogen or (ii) a modification with a protecting group selected from the group consisting of acetyl, benzoyl, tosyl, sulfonyl benzene, benzyloxycarbonyle and palmitoyl; wherein R is O(Z) or N(Z′)(Z′), and wherein Z, Z′ and Z′ are independently of each other a hydrogen or a protecting group selected from the group consisting of methyl, ethyl, propyl, phenyl, hexyl, decyl and hexadecyl, or a racemate, an enantiomer or a diastereomer thereof, or a mixture thereof, or a salt thereof. A peptide of formula II (SEQ ID NO: 2): Lip-A-His-B-C-Trp-R (II) wherein: Lip is a lipoyl residue of configuration R or S; His is a histidine residue of configuration L; Trp is a tryptophane residue of configuration L; A is absent, is an amino acid residue of configuration L or D selected from the group consisting of a lysine residue, an alanine residue, a glutamic acid residue and a glycine residue, or is a spacer of formula: NH—(CH2)n-CO— wherein n is an integer comprised between 2 and 14; B is an aromatic amino acid residue of configuration D selected from the group consisting of a phenylalanine residue, a homophenylalanine residue, a tryptophane residue, a β-(1-Naphthyl)-alanine residue, a β-(2-Naphthyl)-alanine residue and a phenylglycine residue; C is a basic amino acid residue of configuration L selected from the group consisting of an arginine residue, a lysine residue, an ornithine residue and a homoarginine residue; wherein R is O(Z) or N(Z′)(Z′),

### CROSS REFERENCE TO RELATED APPLICATIONS

This application claims priority, under 35 U.S.C. §119(e), of U.S. provisional application Ser. No. 60/947,148, filed on Jun. 29, 2007, the content of which is incorporated herein by reference in its entirety.

### FIELD OF THE INVENTION

The present invention relates to novel peptides, use thereof in cosmetic and cosmeceutic applications, and compositions comprising same.

### BACKGROUND OF THE INVENTION

### Cutaneous Aging

Cutaneous aging is a complex phenomenon responsible for progressive changes of the skin, which is due to many intrinsic and extrinsic factors. Genetic, UV exposure, climatic factors (harshness/wind/cold/warm), pollution (chemical, free radicals, contaminant, nitrogen oxide, metals), alcohol consumption and smoking are factors involved in cutaneous aging. More precisely, UV exposure is responsible for epidermis and dermis injuries. Solar UVB (290-315 nm) affects essentially the epidermis, whereas UVA (315-400 nm) reaches mainly the dermis.

Detailed study of histological changes due to UV exposure reveals thickening of the skin, loss of resiliency and decrease in immune functions. Chronic UV radiations cause modification of the dermis biomechanics' properties which make wrinkles appear. Actinic radiance affects epidermis and dermis at different levels. The triggering of elastose process corresponds to the implementation of an abnormal tissue in the upper zone of dermis, which is very characteristic of the chronic action of UVB. This new tissue characterizes itself by a hyperplasia of abnormal elastic fibers and by the occurrence of damaged fibers with the loss of the parallel organization of microfibrils around elastin (Klingman L H, J. Invest. Dermatol 1985-84-272-6).

This process is coupled with an increase of fibers built-up of fibronectin and of fibrillar compounds which are different from elastin.

This tissue remodeling is responsible for the loss of physical chemistry properties of the dermis. Collagen fibers altered by UVB present themselves as dense bundles. UVB radiance whose luminous energy is directly absorbed by the DNA, is mainly leading to changes of the pyrimidic basis.

Analysis of UVA-exposed skin reveals major dermis modifications including collagen modification and accumulation of degenerative tissue, consisting essentially elastic fibers. These modifications manifest themselves as: cutaneous thinning, reduction of elasticity due to radicalar bridging, and changes in cutaneous matrix.

Dermis is the location of major changes as a result of aging. Dermal cells as well as extracellular matrix are altered. Time is responsible for progressive dermis atrophy with rarefaction and disorganization of these major constituents. The quantity of fibrillar collagens, mostly of type I, is reduced leading to bundles thinning without visible modification of their constitutive fibrils.

### UVA and UVB-Induced Aging

UVA-induced photosensitization phenomenons and induction oxidative stress have been proposed as being in part responsible for the deleterious effects of solar light. It is known that UVA is able to induce DNA strand breaks with a low output (Pfaum et al., Carcinogenesis, 1994, 15, 297-300).

Proteins as well as DNA can be the target of reactive species generated by photochemical reactions. Photobiologic works have shown that chronic skin radiation with UVA can increase the division of indissoluble collagen, thus contributing to aging (Klingman et al., Photoderm. Photobiol., 1991, 54, 233-7). In the hyperplasia of elastic fibers, changes of the elastin are observable following UVA radiation.

UVA radiance penetrating more deeply in the skin than UVB, injuries produced are mainly observed at the dermis level. UVA lead essentially to DNA oxidative denaturation, simple or double strand breaks underlying UVA genotoxicity.

The action of UV leads to the build-up of free radicals. Many studies show that reactive oxygen species play an important role in multiple biologic processes. It is known that production of reactive oxygen species lead to chronic mini-inflammations that are involved in aging phenomena. These radical species participate to the modulation of cellular and tissue response, if their production is under cell control. They play a central role in cellular and tissue destruction when the cell looses the control of the production. It is the paradox of oxidative stress.

UVA creates little direct damage to cellular structures. On the other hand, by means of endogenous photosensitization response, it generates reactive oxygen species (ROS). These ROS, as singulet oxygen or superoxyde anion, are powerful aggressors of biological structures. In the cells, they damage the DNA, cell membranes and some proteins of the extracellular matrix. Even if cells are equipped with a complex antioxidant defense system to fight against oxidative stress able to maintain the intracellular redox potential, the capacity of these defense systems is not unlimited and may be transcended by an intense oxidative stress and an overproduction of ROS caused by immoderate exposure to UV, pollution, alcohol consumption or smoking.

The cell possesses various defense systems enabling it to fight against the overproduction of ROS and their consequences. Amongst these defenses systems are the following:

(a) Protection by enzymatic systems such as superoxide dismutase, catalase and glutathioneperoxydase;

(b) Protection by small molecules, including glutathione, lipoic acid, vitamin E (α-tocopherol), vitamin C (ascorbic acid) and carotenoids. Their antioxidant properties are associated to their ability to intercept or deactivate excited or radicalar states. Glutathione belongs to this group of small molecules;

(c) Trapping of metallic ions, namely protection against the activation of peroxides and O₂° system (superoxyde anion)/H₂O₂ (oxygenated water).

In parallel to these defense systems, repair systems exist whose function is to eliminate damaged material that defense systems failed to protect.

### Glutathione

Glutathione is involved at many levels against oxidative stress and thus plays a major role in against aging processes. It is a tripeptide that may interact directly with activated oxygen species. It exists in a reduced form (GSH) and an oxidized form (G-S-S-G) (SEQ ID NO: 14), these forms being in equilibrium in organisms, and plays a fundamental role in the interruption of the radicalar propagation chains. Glutathione can react with hydroxyl radical or singulet oxygen. Thus, glutathione's strong electron-donor capacity combined with its high intracellular concentration confers to it a high reductive power, allowing it to actively participate in the destruction of ROS (The physiological function of glutathion handbook of free radicals and anti-oxydants in biomedicine, Vol II, Boca Raton CRC Press 189/121-32).

Moreover, glutathione recycles antioxidants such as vitamins C and E, restoring their antioxidative power. In the absence of glutathione, other major antioxidants such as vitamin C and E would be incapable of efficiently protecting organisms against oxidative stress. In the presence of an oxidative stress, GSH is generally consumed and transformed in oxidized glutathione (G-S-S-G) (SEQ ID NO: 14). Factors that promote the build-up of reactive oxygen species lead to the consumption of glutathione. The protective role of glutathione (GSH) in UV-induced stress has been observed at different levels of the biological responses.

Tyrell and Pidoux (Photochem. Photobiol., 1988, 47, 405-12) emphasize the protective role of GSH in the damages caused by UV. They show a correlation between sensitivity to UV of human cutaneous fibroblast in culture and the GSH intracellular content. The protective role of thiols was demonstrated by Monet (J. Photochem. Photobiol. B, 1997, 40, 84-90), who showed that GSH rate may be increased in human cutaneous fibroblast in culture treated by different thiols.

### Lipoic Acid

Lipoic acid, another small molecule that is a member of the antioxidant defense system, is active in both in water and lipo-soluble tissues. It exists in an oxidized form (lipoic acid) and in a reduced form (dihydrolipoic acid). It also exists in the form of lipoyl-lysine (Reed, Protein Sci, 1998, 7, 220-224). When the reduced form dihydrolipoic acid neutralizes free radicals and regenerates antioxidants, it is oxidized in lipoic acid (PODDA M. et al., Clin. Dermatol., 2001, 26, 578-82)

When an antioxidant neutralizes a free radical, it looses its antioxidative capacity and becomes pro-oxidant. After neutralization, antioxidants need therefore to be regenerated.

The present description refers to a number of documents, the content of which is herein incorporated by reference in their entirety.

### SUMMARY OF THE INVENTION

The applicant has found that a family of peptides, more particularly peptides of the Formulae I and II below, are useful for preventing, delaying, reducing or treating the effects of aging on skin including photo-aging.

In specific embodiments, the present invention relates to molecules, compositions and methods of acting on specific biological parameters involved in oxidative stress.

In specific embodiments, the present invention relates to an association of lipoic acid and peptidomimetics of human growth factor (HGF) or alpha-MSH.

Surprisingly, Applicants have identified a peptide family having the ability to act on the glutathione regeneration, internal photoprotection against UV-induced DNA modification.

In a first aspect, the present invention provides a peptide comprising a domain of formula I (SEQ ID NO: 1): Lip-A-Gly-His-B-R (I) wherein: Lip is a lipoyl residue of R or S configuration; A is absent or is a lysine residue of configuration L or D; Gly is a glycine residue; His is a histidine residue of configuration L or D; B is a lysine residue of configuration L or D, or a lysine residue of configuration L or D in which the NH₂ group of the side chain comprises a modification, wherein said modification is (i) a replacement with a hydrogen (deamination) or (ii) a modification with a protecting group selected from the group consisting of acetyl, benzoyl, tosyl, sulfonyl benzene, benzyloxycarbonyle and palmitoyl; wherein R is O(Z) or N(Z′)(Z″), and wherein Z, Z′ and Z″ are independently of each other (i.e. may be the same or different) a hydrogen or a protecting group selected from the group consisting of methyl, ethyl, propyl, phenyl, hexyl, decyl and hexadecyl, or a racemate, an enantiomer or a diastereomer thereof, or a mixture thereof, or a salt thereof.

In an embodiment, the above-mentioned peptide has an activity for preventing, delaying, reducing or treating the effects of aging on skin including photo-aging.

In an embodiment, the above-mentioned peptide has a length of 100 amino acids or less. In a further embodiment, the above-mentioned peptide has a length of 90 amino acids or less. In a further embodiment, the above-mentioned peptide has a length of 80 amino acids or less. In a further embodiment, the above-mentioned peptide has a length of 70 amino acids or less. In a further embodiment, the above-mentioned peptide has a length of 60 amino acids or less. In a further embodiment, the above-mentioned peptide has a length of 50 amino acids or less. In a further embodiment, the above-mentioned peptide has a length of 40 amino acids or less. In a further embodiment, the above-mentioned peptide has a length of 30 amino acids or less. In a further embodiment, the above-mentioned peptide has a length of 20 amino acids or less. In a further embodiment, the above-mentioned peptide has a length of 15 amino acids or less. In a further embodiment, the above-mentioned peptide has a length of 10 amino acids or less. In a further embodiment, the above-mentioned peptide has a length of 9 amino acids or less. In a further embodiment, the above-mentioned peptide has a length of 8 amino acids or less. In a further embodiment, the above-mentioned peptide has a length of 7 amino acids or less. In a further embodiment, the above-mentioned peptide has a length of 6 amino acids or less. In a further embodiment, the above-mentioned peptide has a length of 5 amino acids or less.

More specifically, in a accordance with another aspect the present invention there is provided a peptide of formula I (SEQ ID NO: 1): Lip-A-Gly-His-B-R (I) wherein: Lip is a lipoyl residue of R or S configuration; A is absent or is a lysine residue of configuration L or D; Gly is a glycine residue; His is a histidine residue of configuration L or D; B is a lysine residue of configuration L or D, or a lysine residue of configuration L or D in which the NH₂ group of the side chain comprises a modification, wherein said modification is (i) a replacement with a hydrogen (deamination) or (ii) a modification with a protecting group selected from the group consisting of acetyl, benzoyl, tosyl, sulfonyl benzene, benzyloxycarbonyle and palmitoyl; and R is OZ or N(Z′)(Z″), wherein Z, Z′ and Z″ are independently of each other (i.e. may be the same or different) a hydrogen or a protecting group selected from the group consisting of methyl, ethyl, propyl, phenyl, hexyl, decyl and hexadecyl, or a racemate, an enantiomer or a diastereomer thereof, or a mixture thereof, or a salt thereof.

In a specific embodiment of the peptide, A is a lysine residue. In another specific embodiment, B is a lysine residue of configuration L or D. In another specific embodiment, R is NH₂. In another specific embodiment, at least one of the lysine and histidine residues are in configuration L. In another specific embodiment, the lysine and histidine residues are in configuration L. In another specific embodiment, the lipoyl residue is in configuration R. In another specific embodiment, the lipoyl residue is in configuration S. In another specific embodiment, the peptide is Lip-Lys-Gly-His-Lys-NH₂ (SEQ ID NO: 3). In another specific embodiment, the peptide is Lip-Lys-Gly-His-Lys (SEQ ID NO: 4). In another specific embodiment, the peptide is Lip-Gly-His-Lys-NH₂ (SEQ ID NO: 5). In another specific embodiment, the peptide is Lip-Gly-His-Lys (SEQ ID NO: 6). In another specific embodiment, the peptide is Lip-Lys-Gly-His-Lys-N(CH₃)₂ (SEQ ID NO: 7). In another specific embodiment, the peptide is Lip-Gly-His-Lys-N(CH₃)₂ (SEQ ID NO: 8).

In accordance with another aspect of the present invention there is provided a peptide of formula II (SEQ ID NO: 2): Lip-A-His-B-C-Trp-R (II) wherein: Lip is a lipoyl residue of configuration R or S; His is a histidine residue of configuration L; Trp is a tryptophane residue of configuration L; A is absent, is an amino acid residue of configuration L or D selected from the group consisting of a lysine residue, an alanine residue, a glutamic acid residue and a glycine residue, or is a spacer of formula: NH—(CH₂)ₙ—CO— wherein n is an integer comprised between 2 and 14; B is an aromatic amino acid residue of configuration D selected from the group consisting of a phenylalanine residue, a homophenylalanine residue, a tryptophane residue, a β-(1-Naphthyl)-alanine residue, a β-(2-Naphthyl)-alanine residue and a phenylglycine residue; C is a basic amino acid residue of configuration L selected from the group consisting of an arginine residue, a lysine residue, an ornithine residue and a homoarginine residue; and R is OZ or N(Z′)(Z″), wherein Z, Z′ and Z″ are independently of each other a hydrogen or a protecting group selected from the group consisting of methyl, ethyl, propyl, phenyl, hexyl, decyl and hexadecyl, or a racemate, an enantiomer or a diastereomer thereof, or a mixture thereof, or a salt thereof.

In a specific embodiment of the peptide, the lipoyl residue is in configuration R. In another specific embodiment, the lipoyl residue is in configuration S. In another specific embodiment, A is absent. In another specific embodiment, B is a phenylalanine residue. In another specific embodiment, C is an arginine or an ornithine residue. In another specific embodiment, C is an arginine residue. In another specific embodiment, R is NH₂. In another specific embodiment, said peptide is Lip-His-DPhe-Arg-Trp-NH₂. In another specific embodiment, said peptide is Lip-Lys-His-DPhe-Arg-Trp-NH₂. In another specific embodiment, said peptide is Lip-His-Trp-Arg-Trp-NH₂. In another specific embodiment, said peptide is Lip-Lys-His-Trp-Arg-Trp-NH₂. In another specific embodiment, said peptide is Lip-His-DPhe-Orn-Trp-NH₂.

In accordance with another aspect of the present invention there is provided a composition comprising an effective amount of the peptide of the present invention, and a topically, cosmetically or pharmaceutically acceptable excipient or carrier. In a specific embodiment, said effective amount is between about 10⁻⁸ M to about 10⁻² M. In another specific embodiment, said effective amount is between about 10⁻⁶M to about 10⁻⁵ M. In another specific embodiment, said composition is a topical composition. In another specific embodiment, said composition is an aqueous solution, a cream, a water-in-oil emulsion, a oil-in-water emulsion, a gel, a spray, an ointment, a lotion, or a paste. In another specific embodiment, the composition further comprises at least one additional active agent. In another specific embodiment, said at least one additional active agent is a UV filter. In another specific embodiment, said at least one additional active agent is another peptide of Formula I or II.

In accordance with another aspect of the present invention there is provided a use of the peptide of the present invention, or the composition of the present invention, for preventing, reducing, delaying or treating a skin condition.

In accordance with another aspect of the present invention there is provided a use of the peptide of the present invention, or the composition of the present invention, for the preparation of a medicament for preventing, reducing, delaying or treating a skin condition.

In a specific embodiment, said skin condition is an aging-related skin condition. In another specific embodiment, said aging-related skin condition is the appearance or presence of (a) wrinkles, (b) fine lines or (c) both (a) and (b), on the skin. In another specific embodiment, said skin condition is a skin injury. In another specific embodiment, said skin injury is associated with surgical treatment, dermabrasion, laser treatment or peeling. In another specific embodiment, said skin condition is a photo aging-related skin condition.

In accordance with another aspect of the present invention there is provided a use of the peptide of the present invention, or the composition of the present invention, for photo protecting a biological system against UVA- and/or UVB-induced damages.

In accordance with another aspect of the present invention there is provided a use of the peptide of the present invention, or the composition of the present invention, for the preparation of a medicament for photo protecting a biological system against UVA and/or UVB-induced damages. In a specific embodiment, the damages include UV-induced DNA modifications. In another specific embodiment, the damages include UV-induced oxidative lesions.

In accordance with another aspect of the present invention there is provided a use of the peptide of the present invention, or the composition of the present invention, for inhibiting IL-1α-induced IL-8 production in a biological system.

In accordance with another aspect of the present invention there is provided a use of the peptide of the present invention, or the composition of the present invention, for the preparation of a medicament for inhibiting IL-1α induced IL-8 production in a biological system.

In accordance with another aspect of the present invention there is provided a use of the peptide of the present invention, or the composition of the present invention, for increasing glutathione regeneration in a biological system.

In accordance with another aspect of the present invention there is provided a use of the peptide of the present invention, or the composition of the present invention, for the preparation of a medicament for increasing glutathione regeneration in a biological system.

In accordance with another aspect of the present invention there is provided a use of the peptide of the present invention, or the composition of the present invention, for increasing glutathione scavenging activity in a biological system.

In accordance with another aspect of the present invention there is provided a use of the peptide of the present invention, or the composition of the present invention, for the preparation of a medicament for increasing glutathione scavenging activity in a biological system.

In accordance with another aspect of the present invention there is provided a use of the peptide of the present invention, or the composition of the present invention, for preventing, reducing, delaying or treating free radicals-induced inflammation in a biological system.

In accordance with another aspect of the present invention there is provided a use of the peptide of the present invention, or the composition of the present invention, for the preparation of a medicament for preventing, reducing, delaying or treating free radicals-induced inflammation in a biological system.

In a specific embodiment of uses of the present invention, said biological system is a cell, a tissue or an organ. In another specific embodiment, said cell is a skin cell. In another specific embodiment, said organ is skin.

In accordance with another aspect of the present invention there is provided a method of preventing, reducing, delaying or treating a skin condition in a biological system, said method comprising administering an effective amount of the peptide of the present invention, or the composition of the present invention, to said biological system. In a specific embodiment, said skin condition is an aging-related skin condition. In another specific embodiment, said aging-related skin condition is the appearance or presence of (a) wrinkles, (b) fine lines or (c) both (a) and (b), on the skin. In another specific embodiment, said skin condition is a skin injury. In another specific embodiment, said skin injury is associated with surgical treatment, dermabrasion, laser treatment or peeling. In another specific embodiment, said skin condition is a photo aging-related skin condition.

In accordance with another aspect of the present invention there is provided a method of photo protecting a biological system against UVA- and/or UVB-induced damages, said method comprising administering an effective amount of the peptide of the present invention, or the composition of the present invention, to the biological system. In a specific embodiment, the damages include UV-induced DNA modifications. In another specific embodiment, the damages include UV-induced oxidative lesions.

In accordance with another aspect of the present invention there is provided a method of inhibiting IL-1α-induced IL-8 production in a biological system, said method comprising administering an effective amount of the peptide of the present invention, or the composition of the present invention, to said biological system.

In accordance with another aspect of the present invention there is provided a method of increasing glutathione regeneration in a biological system, said method comprising administering an effective amount of the peptide of the present invention, or the composition of the present invention, to said biological system.

In accordance with another aspect of the present invention there is provided a method of increasing glutathione scavenging activity in a biological system, said method comprising administering an effective amount of the peptide of the present invention, or the composition of the present invention, to said biological system.

In accordance with another aspect of the present invention there is provided a method of preventing, reducing, delaying or treating free radicals-induced inflammation in a biological system, said method comprising administering an effective amount of the peptide of the present invention, or the composition of the present invention, to said biological system.

In a specific embodiment of methods of the present invention, said biological system is a cell, a tissue or an organ. In another specific embodiment, said cell is a skin cell. In another specific embodiment, said organ is skin.

In accordance with another aspect of the present invention there is provided a kit or package comprising the peptide of the present invention, or the composition of the present invention, together with instructions for preventing, reducing, delaying or treating a skin condition in a subject. In a specific embodiment, the kit further comprises a solar filter.

In accordance with another aspect of the present invention there is provided a kit or package comprising the peptide of the present invention, or the composition of the present invention, and a container.

In accordance with another aspect of the present invention there is provided a composition for (i) preventing, reducing, delaying or treating a skin condition in a subject, (ii) preventing, reducing, delaying or treating free radicals-induced inflammation in a biological system, (iii) photo protecting a biological system against UVA- and/or UVB-induced damages (iv) increasing glutathione scavenging activity in a biological system (v) increasing glutathione regeneration in a biological system (vi) inhibiting IL-1α-induced IL-8 production in a biological system or (vii) any combination of (i) to (vi), said composition comprising the above-mentioned peptide and a topically, cosmetically or pharmaceutically acceptable excipient or carrier.

In accordance with another aspect of the present invention there is provided the above-mentioned peptide for (i) preventing, reducing, delaying or treating a skin condition in a subject, (ii) preventing, reducing, delaying or treating free radicals-induced inflammation in a biological system, (iii) photo protecting a biological system against UVA- and/or UVB-induced damages (iv) increasing glutathione scavenging activity in a biological system (v) increasing glutathione regeneration in a biological system (vi) inhibiting IL-1α-induced IL-8 production in a biological system or (vii) any combination of (i) to (vi).

As used herein, unless specifically identified, the chirality of amino acid residues of specific peptides described herein are of L configuration. In peptides of the present invention, the terminology -Lys-NH₂

Specific embodiments of the peptides of Formula I of the present invention comprise:

(peptide A1; SEQ ID NO: 3)
1) Lip-Lys-Gly-His-Lys-NH₂;
(peptide A2; SEQ ID NO: 4)
2) Lip-Lys-Gly-His-Lys;
(peptide A3; SEQ ID NO: 5)
3) Lip-Gly-His-Lys-NH₂;
(peptide A4; SEQ ID NO: 6)
4) Lip-Gly-His-Lys;
(peptide A5; SEQ ID NO: 7)
5) Lip-Lys-Gly-His-Lys-N(CH₃)₂;
and
(peptide A6; SEQ ID NO: 8)
6) Lip-Gly-His-Lys-N(CH₃)₂.

Specific embodiments of the peptides of Formula II of the present invention comprise:

```
(peptide B1; SEQ ID NO: 9)
  7) Lip-His-DPhe-Arg-Trp-NH₂;
(peptide B2; SEQ ID NO: 10)
  8) Lip-Lys-His-DPhe-Arg-Trp-NH₂;
(peptide B3; SEQ ID NO: 11)
  9) Lip-His-Trp-Arg-Trp-NH₂;
(peptide B4; SEQ ID NO: 12)
10) Lip-Lys-His-Trp-Arg-Trp-NH₂;
and
(peptide B5; SEQ ID NO: 13)
11) Lip-His-DPhe-Orn-Trp-NH₂.
```

The peptides in Formula I or II may have one or more asymmetrical carbon atoms in enantiomeric or diastereoisomeric form. Accordingly, the present invention provides enantiomers and diastereoisomers and their mixtures, including racemic mixtures, of the peptide of Formula I or II.

The amino acids in the peptides of the present invention may be present in their natural L-configuration, unnatural D-configuration, or as a racemic mixture (DL).

The peptides of Formula I or II of the present invention may be effectively obtained through classical chemical synthesis or by enzymatic synthesis through processes known to persons skilled in the art.

In accordance with the invention, peptides of Formula I or II can be prepared following chemical synthesis processes in solution or on a solid support, e.g., synthesis on a support with resin. Among the resins that lend themselves to this use are Rink resin (or 4-(2′,4′-dimethoxyphenyl-Fmoc-aminomethyl)-phenoxy resin) (H. Rink, Tetrahedron Let., 1987, 28, 3787) and MBHA resin (or 4-methyl-benzhydrylamine resin) (G. R. Matsueda et al., Peptides, 1981, 2, 45).

The initial products obtained are usually protected amino acids. The protective groups can be an acetyl (Ac) group or a 9-fluorenyl-methoxycarbonyl (Fmoc) group on the primary amino function, a tert-butyloxycarbonyl (Boc) group, a Trityl (Trt) group, and a 2,2,5,7,8-pentamethylchromane-6-sulfonyl (Pmc) group on lateral chain functions. Techniques and methods for washing, coupling and deprotecting amino acids/peptides are well known in the art. The peptide thus obtained may be analyzed using techniques well known in the art, e.g., High Performance Liquid Chromatography (HPLC) and mass spectroscopy.

The peptides of the present invention may be modified using methods well known in the art, e.g., to increase their stability and/or to facilitate their uptake/absorption and/or to improve any other desirable characteristic or property of the peptides that is known to one of skill in art. For example, the peptides can be cyclized, charges on the peptides may be neutralized, and the peptides may be linked to other chemical moieties.

The above-mentioned peptides may take the form of a salt prepared from any physiologically acceptable acid, organic or inorganic. In an embodiment, the above-mentioned salt is one that stabilizes the peptide and is tolerated by the skin. In an embodiment, the above-mentioned salt is an acetate salt.

In another aspect, the present invention provides a composition (e.g., a cosmetic, dermatological, nutraceutical, food or pharmaceutical composition), or a food supplement, comprising a peptides of Formula I or II, or a salt thereof.

According to specific embodiments of the present invention, peptides may be used in the preparation of cosmetic and/or dermatologic compositions as a regenerating agent for glutathione.

According to specific embodiments of the present invention, peptides can also be used in the preparation of cosmetic and/or dermatologic compositions as a DNA photo protecting agent.

Specific embodiments of the present invention concern the cosmetic use of a peptide of Formula A or B in a composition as a glutathione regenerator agent and an internal photo protector for UV-induced DNA modification to prevent, delay and/or treat age-related skin symptoms.

Peptides and their salts may be administered for their cosmetic and/or dermatologic use by different routes. The topical route of administration is preferred. They may also be used as food complements and nutraceuticals and be administered topically.

The present invention encompasses methods administering the peptide in an effective amount to provide a desired result. When the peptide of the present invention is used topically for instance, peptide of formula A or B may be present in a concentration between 10⁻⁸ M and 10⁻² M, preferably 10⁻⁵ M et 10⁻⁶ M. In another embodiment, the peptide of formula A or B is present in a concentration between about 0.5 mg/kg to about 50 mg/kg (i.e. 0.5 to 50 PPM or 0.88×10⁻⁶M to 0.88×10⁻⁴M) in the composition of the present invention.

Cosmetic, dermatologic or pharmaceutical compositions may contain, in combination with a peptide of the present invention, any other active capable of preventing, delaying, reducing and/or treating skin aging such as solar filters.

Although according to specific embodiments the compositions of the invention are intended for preventing, delaying and/or treating photo aging, they are also destined to treat skins affected by cosmetic or therapeutic skin treatments.

The present innovation also concerns a composition comprising at least a UVB and/or a UVA filter, organic or mineral, soluble or indissoluble.

The peptides of the present invention may be formulated in a topically applicable cosmetic composition (e.g., a topical formulation). Non-limitative examples of such topically applicable compositions include skin care cream, cleansing cream, ointment, skin care lotion, skin care gel, skin care foam, sun care composition, make-up removal cream, make-up removal lotion, foundation cream, liquid foundation, bath and shower preparation, deodorant composition, antiperspirant composition, shaving products composition, after-shave gel or lotion, beauty aids composition, depilatory cream, soap composition, hand cleaner composition, cleansing bar, baby care, hair care, shampoo, setting lotion, treatment lotion, hair cream, hair gel, coloring composition, restructuring composition, permanent composition, anti-hair loss composition, or any other composition which is adapted for the use in a topical cosmetic regimen.

Creams, as is well known in the arts of pharmaceutical and cosmeceutical formulation, are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solutes) in a liquid such that the molecules of the dissolved substance are dispersed among those of the solvent. The solution may contain other cosmeceutically acceptable chemicals to buffer, stabilize or preserve the solute. Common examples of solvents used in preparing solutions are ethanol, water, propylene glycol or any other cosmeceutically acceptable vehicles.

Gels are semisolid, suspension-type systems. Single-phase gels contain organic macromolecules distributed substantially uniformly throughout the carrier liquid, which is typically aqueous, but also, preferably contain an alcohol, and, optionally, an oil. “Organic macromolecules,” i.e., gelling agents, are crosslinked acrylic acid polymers such as the “carbomer” family of polymers, e.g., carboxypolyalkylenes that may be obtained commercially under Carbopol™. Other examples are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Ointments are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, Pa.: Mack Publishing Co., 1995), at pages 1399 1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy (supra) for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Formulations may also be prepared with liposomes, micelles, and microspheres. Liposomes are microscopic vesicles having a lipid wall comprising a lipid bilayer, and, in the present context, encapsulate one or more components of the anti-aging formulations. Liposomal preparations herein include cationic (positively charged), anionic (negatively charged), and neutral preparations. Cationic liposomes are readily available. For example, N[1-2,3-dioleyloxy)propyl]-N,N,N-triethylammonium (DOTMA) liposomes are available under the tradename Lipofectin™ (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl-8 dodecyl ether, polyoxyl-12 dodecyl ether, nonoxynol 10, and nonoxynol 30.

Microspheres, similarly, may be incorporated into the present formulations. Like liposomes and micelles, microspheres essentially encapsulate one or more components of the present formulations. They are generally although not necessarily formed from lipids, preferably charged lipids such as phospholipids. Preparation of lipidic microspheres is well known in the art and described in the pertinent texts and literature.

In an embodiment, the composition of the present invention further comprises at least one additional active ingredient/agent. In a further embodiment, the above-mentioned at least one additional active ingredient modulate(s) at least one of cell differentiation, cell metabolic activity, cell structure, cell proliferation, extracellular processes and pigmentation.

The composition of the present invention may further comprise at least one of an agent that modulates cell differentiation or proliferation, an anesthesic agent, anti-acne agent, anti-aging agent, antibacterial agent, anticellulite agent, antifungal agent, anti-inflammatory agent, anti-irritant agent, antioxidant agent, antiparasitic agent, antipollution agent, antipruritic agent, anti-rosacea agent, anti-seborrhea agent, anti-stress agent, anti-telangiectasia agent, antiviral agent, anti-wrinkle agent, baby care agent, bath and body agent, calming agent, cleansing agent, collagen synthesis agent, elastase inhibitory agent, exfoliant agent, facial peeling agent, firming agent, foot care agent, free radical scavenging agent, immune function modulator agent, keratolytic agent, lift agent, make-up remover agent, melanogenesis stimulator agent, hair care agent, matrix metalloproteinase inhibitory agent, moisturizing agent, oil absorbent agent, osmoregulator agent, anti-photoaging agent, protecting agent, rejuvenating agent, regenerating agent, restructuring agent, sensitive skin agent, shaving product agent, skin defense enhancer agent, skin clarifier agent, skin repair agent, slimming agent, smoothing agent, softening agent, soothing agent, sun care agent, sunless tanning agent, tensing agents and whitening agent, or any other agent adapted for use in a cosmetic regimen that comprises topical application of said cosmetic composition, and which complements or supplements the effect of the peptide of the present invention.

Without being so limited, agents that modulate cell differentiation or proliferation include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), vitamin D and its derivatives (cholecalciferol, ergocalciferol, 25-hydroxycholecalciferol), growth factors and estradiol derivatives.

Without being so limited, anaesthesics include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include lidocaine chlorhydrate and its derivatives.

Without being so limited anti-acne agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include benzoyl peroxide, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), salicylic acid, sulfur, sulfurated lime, alcohol and acetone.

Without being so limited, anti-aging/anti-wrinkle agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include hyaluronic acid, sodium-2-pyrrolidone carboxylate, glycosaminoglycans, kinetin, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), epidermal growth factor, ceramide, ethylbisiminomethylguaiacol manganese chloride, glycation inhibitors, chrysanthellum indicum extract and aphanizomenon flos aquae extract.

Without being so limited, antibacterial agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include eucalyptus extract, clindamycin phosphate, cavacrol, erythromycin and antibiotics belonging to the group of tetracyclines.

Without being so limited, antifungal agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include econazole, ketoconazole, miconazole, amphotericin B, terbinafine and octopirox.

Without being so limited, anti-inflammatory agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include allantoin, vitamin E and its derivatives (α-tocopherol, δ-tocopherol, γ-tocopherol), chamomile oil, gingko biloba oil and camellia sinensis extract.

Without being so limited, anti-irritant/soothing/smoothing/calming agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include allantoin, camellia sinensis extract, lavender oil, aloe vera, linden extract, epilobium angustifolium extract, chysanthellum indicum extract, cola nitida extract and alteromonas ferment extract.

Without being so limited, antioxidant agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include furfuryladenine, panthenol, lipoic acid, ubiquinone, niacinamide, melatonin, catalase, glutathione, superoxide dismutase, polyphenols, cysteine, allantoin, kinetin, vitamin C and its derivatives (ascorbyl palmitate, magnesuim ascorbyl phosphate, sodium ascorbyl phosphate), vitamin E and its derivatives (α-tocopherol, δ-tocopherol, γ-tocopherol), grape seed extract and camellia sinensis extract.

Without being so limited, antipruritic agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include thenaldine, trimeprazine, cyproheptadine.

Without being so limited, anti-rosacea/anti-telangiectasia agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include metronidazole, vasoconstrictors, benzoyl peroxide, azelaic acid, sulphur, soy proteins and glycosaminoglycans.

Without being so limited, anti-seborrhea agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include progesterone derivatives, isoleutrol and hinokitiol.

Without being so limited, sensitive skin agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include rose oil and jasmine oil.

Without being so limited, cleansing agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include ammonium lauryl sulfate, ammonium laureth sulfate, cocamide MEA, triethanolamine lauryl sulfate, sodium stearate and nettle leaf extract.

Without being so limited, collagen synthesis agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), vitamin C and its derivatives (ascorbyl palmitate, magnesium ascorbyl phosphate, sodium ascorbyl phosphate), growth factors and its derivatives.

Without being so limited, exfoliant agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include alpha/beta hydroxy acids, salicylic acid, glycolic acid, lactic acid, citrus acid and walnut shell powder.

Without being so limited, facial peeling agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include glycolic acid, lactic acid, trichloroacetic acid and phenol.

Without being so limited, firming/tensing agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include dimethylaminoethanol, neuro-cosmetic actives (Botox™-like), chitosan, arnica extract, fennel-sweet oil and papaya extract.

Without being so limited, free radical scavenging/antipollution/anti-stress agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include grape seed extract, alpha-tocopherol and the esters thereof, superoxide dismutase, some chelating agents of metals, vitamin C and its derivatives (ascorbyl palmitate, magnesium ascorbyl phosphate, sodium ascorbyl phosphate).

Without being so limited, hair care agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include poly-D-glucosamine, poly-N-acetyl-D-glucosamine, stearalkonium chloride and triethanolamine lauryl sulfate.

Without being so limited, matrix metalloproteinase inhibitory agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include camellia sinensis extract, polyphenols, spatholobi caulis extract, euonymus alatus extract, rhizoma notopterygii extract, quercetin, glycosaminoglycans, polymethoxy flavonoid, N-acetyl-cysteine, 2-furildioxime, isoflavone, vitamin C and its derivatives (ascorbyl palmitate, magnesium ascorbyl phosphate, sodium ascorbyl phosphate), retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester) and hydroxamate derivatives.

Without being so limited, moisturizing agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include cucumber extract, sodium-2-pyrrolidone carboxylate, sodium PCA, sodium hyaluronate, chitin and its derivatives, alpha hydroxy acids, hyaluronic acid and hydrolysed wheat protein.

Without being so limited, osmoregulator agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include mannitol, dulcitol and betaine.

Without being so limited, protecting agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include poly-N-acetyl-D-glucosamine, poly-D-glucosamine, alkyloamides, chitosan, chrysanthellum indicum extract, camellia sinensis extract and alteromonas ferment extract.

Without being so limited, rejuvenating agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include rosemary extract, rosewood extract, geranium extract and vitamin E and its derivatives (α-tocopherol, δ-tocopherol, γ-tocopherol).

Without being so limited, skin repair agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), allantoin, eucalyptus extract, lavender oil, rose oil and activators of collagen synthesis and activators of components of the skin's extracellular matrix.

Without being so limited, slimming/anticellulite agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include chrysanthellum indicum extract, dihydromyricetin, theobromine, theophylline, aminophylline, caffeine, isopropylarterenol hydrochloride, epinephrine, α-MSH agonists, adenylate cyclase activators and phosphodiesterase inhibitors.

Without being so limited, sun care/photo aging agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include PABA (p-aminobenzoic acid) and derivatives, gluconolactone, salicylates, cinnamates, benzophenones, dibenzoylmethanes, oxybenzone, vitamin E and its derivatives (α-tocopherol, δ-tocopherol, γ-tocopherol), ethylbisiminomethylguaiacol manganese chloride, glycosaminoglycans, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), titanium dioxide, octyl methoxycinnamate, benzophenone, octyl salicylate, epilobium angustifolium extract, rumex occidentalis extract, chrysanthellum indicum extract, camellia sinensis extract and alteromonas ferment extract.

Without being so limited, sunless tanning/melanogenesis stimulator agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include dihydroxyacetone, α-MSH agonists, adenylate cyclase activators and phosphodiesterase inhibitors.

Without being so limited, toning agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include nettle extract, orange blossom extract, rosewood extract and witch hazel extract.

Without being so limited, whitening/pigmentation agents include plant extracts, algae extracts, fruit extracts, vegetable extracts, leguminous plant extracts, ferments, proteolytic hydrolysates, peptides, yeast extracts and its derivatives, microorganism extracts, animal derivative extracts and synthetic compounds. More particularly, such agents include arbutin, azealeic acid, vitamin C and its derivatives (ascorbyl palmitate, magnesuim ascorbyl phosphate, sodium ascorbyl phosphate), hydroquinone, N-acetyl-4-S-cysteanimylphenol, kojic acid, melanostat (melanostatine), tretinoin, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), ruminex occidentalis extract, licorice, mulberry, arctostaphylos uva-ursi (bearberry), tyrosinase inhibitors, melanosome-transfer inhibitors and melanin scavengers.

In an embodiment, the composition of the present invention further comprises a pharmaceutically acceptable topical carrier, vehicle, excipient or additives (i.e. topically/cosmetically acceptable carrier, vehicle, excipient or additives). Such carrier, vehicle, excipient or additives are well known in the art and may be used, for example, to improve final formulation regarding organoleptic properties, skin penetration and accessibility of the active ingredient. Examples of carriers, vehicles or excipients include: buffering agent, carrier agent, chelating agent, conditioner agent, coloring agent, detackifier agent, emollient agent, emulsifier agent, film former agent, foaming agent, humectant agent, lactylate agent, lipophilic agent, lubricant agent, neutralizer agent, oil agent, opacifier agent, preservative agent, solubilizer agent, solvent agent, stabilizer agent, surfactant agent, thickener agent, viscosity agent, water absorbent agent, wetting agent, perfume and thermal water.

The composition of the present invention may be formulated so as to provide for a specifically controlled delivery system. Non-limitative examples of such delivery systems include slow delivery system, rapid delivery system, immediate delivery system, delayed delivery system, zero-order delivery system and dual or multiple speed delivery system. Such controlled delivery systems may be achieved with specific formulations including chemical delivery systems, multiple emulsions, microemulsions, nanoemulsions, encapsulations such as liposomes, microspheres, nanospheres, microsponges, beads and cyclodextrins, polymeric matrices, polymeric cosmetic conjugates, oil body/oleosin, oil-soluble molecular film, skin patches, unit dosages.

Without being so limited, buffering agents are salts of bases/acids, compatible with the nature of the skin and with its pH. Sodium acetate is an example of a frequently used buffer agent.

Without being so limited, carrier agents are ingredients capable of aiding the application of the active ingredient. Isohexadecane is an example of a frequently used carrier.

Without being so limited, chelating agents are ingredients capable of binding mono and divalent cations, such as tetrasodium EDTA and disodium EDTA.

Without being so limited, conditioner agents are ingredients with lubricating action and hydrating effect, such as cetrimonium chloride, dicetyldimonium chloride, trideceth-I2, quaternium-Z7, quaternium-I8, polyquaternium-10, behentrimonium methosulfate, cetearyl alcohol, stearamidopropyl dimethylamine, trimethylsilylamodimethicone, isolaureth-6, octoxynol-4, dimethicone, dimethiconol, cyclopentasiloxane, pareth-7, pareth-9, linoleic acid and glycerin.

Without being so limited, detackifier agents are ingredients capable of adsorbing onto tacky materials and reduce their tendency to adhere, such as cyclopentasiloxane, dimethicone and vinyl dimethicone, phenyl trimethicone, isopropyl esters, isostearate esters, dimethyl sebacate and dipropyl sebacate.

Without being so limited, emollient agents are ingredients with lubricating action and hydrating effect, such as isopropyl palmitate, sunflower seed oil, mineral oil, stearyl stearate, isopropyl myristate, lanolin, caprylic, capric triglyceride, cyclopentasiloxane, dimethicone, vinyl dimethicone, bis-phenylpropyl dimethicone, alkyl dimethicone, sorbitan stearate, sucrose distearate, myristyl alcohol, myristyl lactate, cetyl acetate, dicaprylyl ether, floraester-20, maleated soybean oil, cyclomethicone, shea butter, hydrogenated coconut oil, isopropyl palmitate, diisostearoyl trimethylolpropane siloxy silicate and alkyl benzoate.

Without being so limited, emulsifier agents are ingredients capable of preventing the separation of immiscible substances in an emulsion, of helping to distribute evenly one substance in another, of improving texture, homogeneity, consistency and stability, such as cetearyl alcohol, glyceryl stearate, alkyl acrylate crosspolymer, stearic acid, emulsifying wax, sorbitan oleate, sorbitan stearate, polysorbate, polyethylene glycopolysorbate, triethanolamine, cyclopentasiloxane, dimethicone copolyol, PEG-30 dipolyhydroxystearate, sucrose distearate, PEG-100 stearate, sodium dioctylsulfosuccinate, polyacrylamide, isoparaffin, laureth-7, cetyl phosphate, DEA cetyl phosphate, glycol stearate, stearyl alcohol, cetyl alcohol, behentrimonium methosulfate and ceteareth-2.

Without being so limited, film former agents are ingredients capable of forming a dimensionally stable and continuous film to minimize the formula tackiness, such as wheat protein, eicosene copolymer, perfluoromethylisopropyl ether, diisostearoyl trimethylolpropane siloxy silicate, trimethylsiloxysilicate, dimethicone, vinyl dimethicone and cyclopentasiloxane.

Without being so limited, foaming agents are ingredients capable of regulating the amount of air in a product, such as lauramide DEA and cocamide MEA, disodium laureth sulfosuccinate, disodium N-octadecyl sulfosuccinamate, ammonium lauryl sulphate, triethanolamine lauryl sulfate, sodium lauryl sulphate and sodium 2-ethylhexylsulfate.

Without being so limited, humectant agents are ingredients capable of maintaining constant humidity and retaining moisture, such as glycerine, PEG-8, butylene glycol and propylene glycol.

Without being so limited, lubricant agents are ingredients capable of adding slipperiness and reducing friction to improve application, such as dimethicone and dimethicone copolyol.

Without being so limited, neutralizer agents are ingredients capable of changing the acid-alkaline balance, such as triethanolamine and sodium hydroxide.

Without being so limited, opacifier agents are ingredients capable of changing the look of a clear or translucent product to a creamier or pearlier one, such as glyceryl stearate and PEG-100 stearate.

Without being so limited, preservative agents are ingredients capable of retarding or preventing microbial or chemical spoilage and protecting against discoloration, such as DMDM hydantoin, methylparaben, propylparaben, phenoxyethanol, ethylparaben, butylparaben, imidazolidinyl urea, diazolidinyl urea, quaternium-8, quaternium-14, quaternium-15, propylene glycol, dehydroacetic acid, methylchloroisothiazolinone, methylisothiazolinone and germaben.

Without being so limited, solubilizer agents are ingredients capable of allowing incompatible ingredients to become part of a homogeneous solution, such as polysorbate, ceteareth, steareth and PEG.

Without being so limited, stabilizer agents are ingredients capable of maintaining physical and chemical properties during and after processing, preventing or limiting changes in the physical properties of a substance during product life, such as polyethylene, sodium chloride, stearyl alcohol, xanthan gum, tetrasodium EDTA and dimethicone copolyol.

Without being so limited, surfactant agents are ingredients capable of reducing surface tension when dissolved in water or a water solution, reducing interfacial tension between two liquids or between a liquid and a solid, such as sodium dioctylsulfosuccinate, octoxynol-40, isolaureth-6, ammonium lauryl sulfate, lauryl alcohol, lauramide DEA and cocoamidopropyl betaine.

Without being so limited, thickener agents are ingredients capable of absorbing water to impart body, improve the consistency or texture, and stabilize an emulsion, such as stearic acid, magnesium aluminum silicate, carbomer, alkyl acrylate crosspolymer, polyacrylamide, isoparaffin, laureth-7, cetyl alcohol, xanthan gum, alkyl dimethicone, hydroxyethylcellulose, glyceryl stearate, pentaerythrityl tetrastearate, stearyl alcohol and polyquaternium-10.

Without being so limited, viscosity agents are ingredients capable of controlling the degree of fluidity and the internal resistance to flow exhibited by a fluid, such as magnesium aluminum silicate, caprylyl glycol and myristyl alcohol.

Without being so limited, water absorbent agents are ingredients capable of absorbing the product's water to maintain the moisture, such as carboxyvinyl polymer, acrylic copolymer, polyacrylamide, polysaccharides, natural gum, clay, modified clay, metallic salt and fatty acid.

Without being so limited, wetting agents are ingredients capable of reducing the surface tension of the water for better penetration or spread over the surface, such as caprylate, caprylyl glycol, glyceryl caprate, polyglyceryl-2 caprate, polyglyceryl-6, polyglyceryl-3 laurate and TEA-laureth sulfate.

The peptide or composition of the present invention may be packaged in any suitable manner, including but not limited to, a jar, a bottle, a tube, a stick, a roller-ball applicator, an aerosol spray device, etc., in the conventional manner. The peptide or composition of the present invention could be packaged as a kit of two or more separate compartments, including one containing the active ingredients and a second containing a topically/dermatologically-acceptable vehicle, which may be mixed together at some fixed time point prior to application. For example, the active ingredients, in the form of a cream, a powder, a tablet, a capsule or a liquid, may be contained in sealed, single-use packets, which may be opened and mixed with the topically-acceptable vehicle, which may also be stored in pre-measured form in sealed, single-use packets. Alternatively, the active ingredients and the topically-acceptable vehicle may be provided in larger quantities from which the needed amount could be withdrawn using various measuring devices, such as a measuring spoon or cup for solids, or a calibrated vial or dropper for liquids. The peptide or composition of the present invention may be spread onto a substrate and then subsequently packaged. Suitable substrates include dressings, including film dressings, and bandages. In an embodiment, the kit or package may comprise instructions for use/application, e.g., instructions for preventing, reducing, delaying or treating a skin condition.

In another aspect, the present invention provides the use (e.g., cosmetic or therapeutic use) of a peptide of formula I or II for preventing, reducing, delaying or treating a skin condition in a subject.

In an embodiment, the above-mentioned skin condition is an aging-related skin condition (i.e. intrinsic aging or extrinsic aging (e.g. sun damages) of the skin. The aging-related skin condition may, for example, involve wrinkles, fine lines, age spots, sun damage (particularly UV radiation-induced oxidative stress), blemishes, hyperpigmented skin, age spots, increased skin thickness, loss of skin elasticity and collagen content, dry skin, lentigines, and/or melasmas or any combination thereof. In an embodiment, the above-mentioned aging-related skin condition is the appearance or presence of (a) wrinkles, (b) fine lines or (c) both (a) and (b), on the skin.

In another embodiment, the above-mentioned skin condition is skin damage caused by a cosmetic or therapeutic treatment or by an injury (e.g., a surgical intervention involving the skin, laser treatment of the skin, dermabrasion or peeling (e.g., to assist in the healing process).

In an embodiment, the above-mentioned biological system is a cell or cells, a tissue, an organ or a subject. In a further embodiment, the above-mentioned cell or cells is/are a skin cells such as a fibroblast, or a combination of cells including fibroblasts. In another embodiment, the above-mentioned organ is skin.

The method of delivery of the peptide or composition of the present invention may vary, but usually involves application to an area of skin prone to, or affected by, an aging-related skin condition, e.g., any skin condition or disorder associated with, caused by, or affected by, intrinsic aging and/or extrinsic aging. The aging-related skin condition may, for example, involve wrinkles, fine lines, age spots, sun damage (e.g., UV radiation-induced oxidative stress), blemishes, hyperpigmented skin, increased skin thickness, loss of skin elasticity and collagen content, dry skin, lentigines, and/or melasmas.

A cream, lotion, gel, ointment, paste or the like may be spread on the affected surface and gently rubbed in. A solution may be applied in the same way, but more typically will be applied with a dropper, swab, or the like, and carefully applied to the affected areas.

The application regimen will depend on a number of factors that may readily be determined, such as the severity of the condition and its responsiveness to initial treatment, but will normally involve one or more applications per day on an ongoing basis. One of ordinary skill may readily determine the optimum amount of the formulation to be administered, administration methodologies and repetition rates. In general, it is contemplated that the formulations of the invention will be applied in the range of once or twice weekly up to once or twice daily.

In an embodiment, the above-mentioned subject is a mammal. In a further embodiment, the above-mentioned mammal is a human.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 is a graphic presenting the antioxidative effect of peptide A1 (CGF03) (SEQ ID NO: 3) on UVA irradiated NCTC 2544 in culture;

FIG. 2 is a graphic presenting the antioxidative effect of peptide B1 (RE100) (SEQ ID NO: 9) on UVA irradiated NCTC 2544 in culture; and

FIG. 3 is a graphic presenting the effect of peptide B1 (SEQ ID NO: 9) on glutathione regeneration measured by capture percentage of radicals.

### DESCRIPTION OF ILLUSTRATIVE EMBODIMENTS

The present invention is illustrated in further details by the following non-limiting examples.

### Example 1

### Synthesis of Lip-Lys-Gly-His-Lys-NH₂

### Peptide A1; SEQ ID NO: 3

Peptide A1 was synthesized on a solid support with a Rink amide resin whose functionalization is between 0.3 and 0.6 mmole/g of resin. The Rink amid resin was first prepared by washing with Dimethylformamide (DMF) (2 washings), then followed by the deprotection step described below. For each amino acid to be coupled, the following steps were repeated: coupling the amino acid, washing the resin, deprotecting the main chain's amino function, and again washing the resin.

Coupling: a mixture of two benzotriazole-1-yl-oxy-tris-(dimethylamino)-phosphonium hexafluorophosphate (BOP) (or 2-(1H-benzotriazol-1-yl) 1,1,3,3-tetramethyluronium hexafluorophosphate, HBTU) equivalents, two diispropylethylamine (DIEA) (or N-methylmorpholine, NMM) equivalents and two 9-fluorenylmethoxycarbonyl (Fmoc)-AA-OH equivalents, was used for 2 hours in DMF.

Washing: two successive DMF washings, one methanol washing, two dichloromethane washings and one DMF washing were performed.

Deprotection: a 80/20 DMF/piperidine mix with 2% ethanediol (to trap radicals), was used once for 3 minutes and then for 7 minutes.

Washing: (same as above).

After the various amino acids have been coupled, the lipoic acid residue was coupled on the N-terminal function of the peptide in the same manner as an amino acid and the resulting peptide was cleaved from the resin using a 50/50 Trifluoroacetic acid (TFA)/dichloromethane mix with 2% ethanediol for 90 minutes. Dichloromethane and TFA were evaporated under a nitrogen flow, followed by precipitation with diethylether and purification by preparative liquid chromatography with a reversed-phase C18 column.

Lip-Lys-Gly-His-Lys-NH₂ (peptide A1) was synthesized using the following protected amino acids: Fmoc-His(Trt)-OH, Fmoc-Lys(Boc)-OH, and Fmoc-Gly-OH.

### Abbreviations:

Pmc: Pentamethylchroman-6-sulfonyl

Fmoc: 9-fluorenylmethoxycarbonyl

TFA: trifluoracetic Acid

DMF: Dimethylformamide

BOP: Hexafluorophosphate of benzotriazole-1-yl-oxy-tris(dimethylamino)-phosphonium

HBTU: Hexafluorophosphate of 2-(1H-benzotriazole-1-yl)1,1,3,3-tetramethyluronium

DIEA: Diisopropyl ethyl amine

NMM: N-Methyl morpholine

The desired peptide was obtained and tested in the following assays for purity and activity. Results were analyzed by High Performance Liquid Chromatography (HPLC) and by Mass Spectrometry.

### HPLC

Stationery Phase: Inverse phase C18 Column of dimensions 4.6×50 mm, 3.5 μm.

Mobile Phase: binary phase of 0.1% TFA in water and 0.1% TFA in acetonitrile.

A 12 minute gradient was applied from 0% to 80% of acetonitrile phase.

Wavelength detection: 214 nm

Mass Spectrometry: Positive Electrospray, with a conic tension of 9V, a source temperature of 120° C. and a scanning duration of 6 seconds.

### Example 2

### Synthesis of Lip-Lys-Gly-His-Lys

### Peptide A2; SEQ ID NO: 4

Peptide A2 was synthesized as generally described in Example 1 with the following adaptations:

Peptide A2 was synthesized using the following protected amino acids: Fmoc-His(Trt)-OH, Fmoc-Lys(Boc)-OH, and Fmoc-Gly-OH.

### Example 3

### Synthesis of Lip-Gly-His-Lys-NH₂

### Peptide A3; SEQ ID NO: 5

Peptide A3 was synthesized as generally described in Example 1 with the following adaptations:

Peptide A3 was synthesized using the following protected amino acids: Fmoc-His(Trt)-OH, Fmoc-Lys(Boc)-OH, and Fmoc-Gly-OH.

### Example 4

### Synthesis of Lip-Gly-His-Lys

### Peptide A4; SEQ ID NO: 6

Peptide A4 was synthesized as generally described in Example 1 with the following adaptations:

Peptide A4 was synthesized using the following protected amino acids: Fmoc-His(Trt)-OH, Fmoc-Lys(Boc)-OH, and Fmoc-Gly-OH.

### Example 5

### Synthesis of Lip-Lys-Gly-His-Lys-N(CH₃)₂

### Peptide A5; SEQ ID NO: 7

Peptide A5 was synthesized as generally described in Example 1 with the following adaptations:

Peptide A5 was synthesized using the following protected amino acids: Fmoc-His(Trt)-OH, Fmoc-Lys(Boc)-OH, and Fmoc-Gly-OH.

### Example 6

### Synthesis of Lip-Gly-His-Lys-N(CH₃)₂

### Peptide A6; SEQ ID NO: 8

Peptide A6 was synthesized as generally described in Example 1 with the following adaptations:

Peptide A6 was synthesized using the following protected amino acids: Fmoc-His(Trt)-OH, Fmoc-Lys(Boc)-OH, and Fmoc-Gly-OH.

### Example 7

### Synthesis of Lip-His-DPhe-Arg-Trp-NH₂

### Peptide B1; SEQ ID NO: 9

Peptide B1 was synthesized as generally described in Example 1 with the following adaptations:

Peptide B1 was synthesized using the following protected amino acids: Fmoc-His(Trt)-OH, Fmoc-DPhe-OH, Fmoc-Arg(Pmc)-OH and Fmoc-Trp-OH.

### Example 8

### Synthesis of Lip-Lys-His-DPhe-Arg-Trp-NH₂

### Peptide B2; SEQ ID NO: 10

Peptide B2 was synthesized as generally described in Example 1 with the following adaptations:

Peptide B2 was synthesized using the following protected amino acids: Fmoc-Lys(Boc)-OH, Fmoc-His(Trt)-OH, Fmoc-DPhe-OH, Fmoc-Arg(Pmc)-OH and Fmoc-Trp-OH.

### Example 9

### Synthesis of Lip-His-Trp-Arg-Trp-NH₂

### Peptide B3; SEQ ID NO: 11

Peptide B3 was synthesized as generally described in Example 1 with the following adaptations:

Peptide B3 was synthesized using the following protected amino acids: Fmoc-His(Trt)-OH, Fmoc-Arg(Pmc)-OH and Fmoc-Trp-OH.

### Example 10

### Synthesis of Lip-Lys-His-Trp-Arg-Trp-NH₂

### Peptide B4; SEQ ID NO: 12

Peptide B4 was synthesized as generally described in Example 1 with the following adaptations:

Peptide B4 was synthesized using the following protected amino acids: Fmoc-Lys(Boc)-OH, Fmoc-His(Trt)-OH, Fmoc-Arg(Pmc)-OH and Fmoc-Trp-OH.

### Example 11

### Synthesis of Lip-His-DPhe-Orn-Trp-NH₂

### Peptide B5; SEQ ID NO: 13

Peptide B5 was synthesized as generally described in Example 1 with the following adaptations:

Peptide B5 was synthesized using the following protected amino acids: Fmoc-His(Trt)-OH, Fmoc-DPhe-OH, and Fmoc-Trp-OH.

### Example 12

### Evaluation of Internal Photo Protection Against UVA and UVB Using Comet Test

The proto protective ability of peptides A1 and B1 against UVA and UVB was evaluated using a Comet test according to the protocol of De Meo (De Meo et al. Mutation Res. 1991; 260; 295-306).

The comet test enables the quantification of DNA strand breaks induced by a genotoxic agent.

Material: Primary culture of normal human melanocytes.

Treatment: Cells were treated for 2 h at 37° C. with peptides A1 and B1 separately at 3×10⁻⁸ M and then exposed to UVA (365 nm, 0.8 J/cm²) or UVB (312 nm/0.06 J/cm²).

Results are reported in Table 1 below as a coefficient of genomic protection against UVA or UVB (CGP %).

TABLE 1

| | CGP (%) |
|---|---|
| Non treated cells | 100% |
| Irradiated cells (UVA) | 0% |
| Irradiated cells (UVB) | 0% |
| Peptide A1 at 3 × 10⁻⁸ M | |
| +UVA | 93% |
| +UVB | 46.2% |
| Peptide B1 at 3 × 10⁻⁸ M | |
| +UVA | 69.9% |
| +UVB | 85.8% |

Peptides A1 and B1 displayed a photo protective effect against UVA and UVB, and protection against UVA- and UVB-induced DNA strand breaks.

### Example 13

### Effect of Peptides A1 and B1 on UVA-Induced Oxydative Lesions on Human Keratinocytes

Material: Keratinocytes NCTC-2544.

Treatment: Keratinocytes were incubated with either peptide (A1 or B1) for 24 h, re-stimulated with the same peptide for 30 minutes and then irradiated with UVA (15 J/cm²).

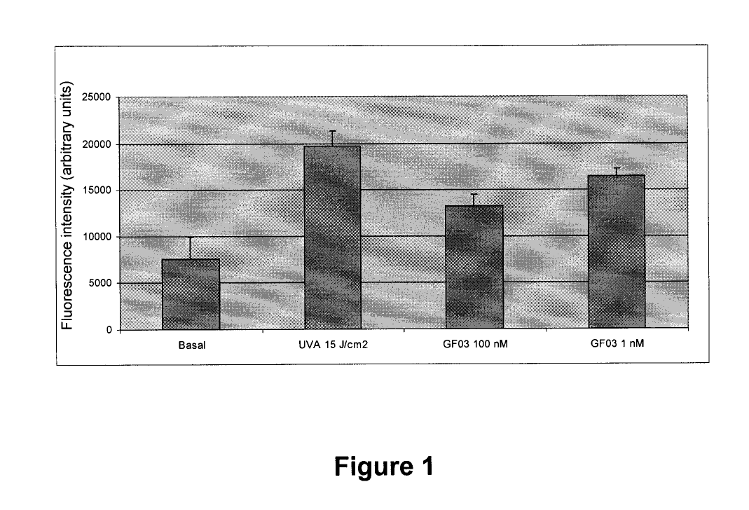

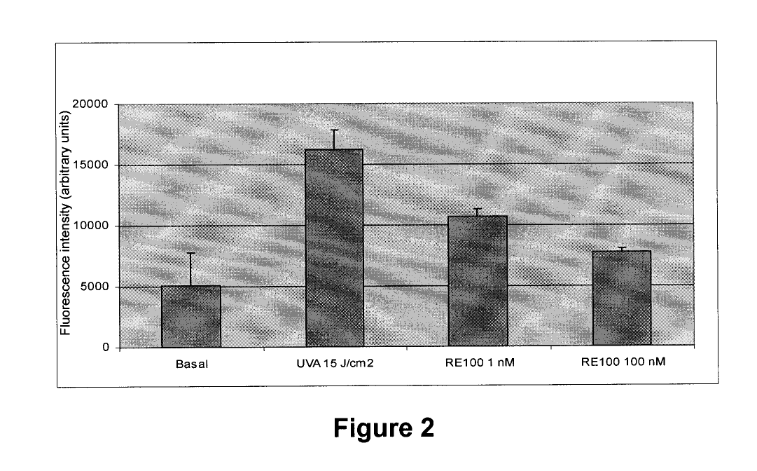

Results: The cells exposed to UVs are subjected to damages/lesions induced by oxidative stress. The oxydative damages or lesions were monitored with the formation of 8-oxo D guanosine and the effect of the peptides was expressed as a percentage of inhibition of the production of oxidative damages/lesions. The results are presented in FIGS. 1 and 2 for peptides A1 (GF03) and B1 (RE100), respectively, and are summarized in Table 2 below.

TABLE 2

| | % INHIBITION |
|---|---|
| Non treated cells | 0% |
| UVA-irradiated cells 15 J/cm² | 100% |
| +peptide A1 (100 nM) | −53% |
| +peptide A1 (1 nM) | −27% |
| +peptide B1 (100 nM) | −76% |
| +peptide B1 (1 nM) | −50% |

### Example 14

### Evaluation of Peptide B1's Anti-Inflammatory Potential Measured by the Inhibition of IL-1α-Induced IL-8 Production

Material: Normal human dermal fibroblasts.

Treatment: Concomitant treatment with peptide B1 at two concentrations (10 pM and 1 pM) and IL-1α for 24 hours followed by IL-8 dosage by ELISA.

Results are presented in Table 3 below.

TABLE 3

INHIBITION of
IL-8 production

| | 10 pM | 1 nM |
|---|---|---|
| Peptide B1 | 56% | 60% |

### Example 15

### Evaluation of Glutathione Regeneration in the Presence of a Superoxide Anion by Fluorometry

Peroxydase (Horseradish peroxydase or HRP) transforms H₂O₂ into O₂^{•-} which reacts on fluorescent scopoletine and transforms it into a non fluorescent species. A rapid decrease in fluorescence is observed as a function of O₂^{•-} release. In the presence of a scavenger for radical O₂^{•-} (such as glutathione), the decrease in fluorescence is less pronounced. (Antioxidant Characterisation, Methodology and Mechanism, Barry Halliwell, Biochemical Pharmacology, 49, 1341-1348, (1995)) and (The scopoletin assay for hydrogen peroxide, a review and a better method, Jean T. Corbett, J. Biochem. Biophys. Methods, 18, 297-308 (1989)).

Material: A fluorometer set at 394 nm (excitation state) with a slot of 6, and 455 nm (emitting state) with a slot of 8 was used. Continuous data collection was made over a period of 180 seconds. The data obtained at the end of 180 seconds was compared to that obtained with a negative control (without scavenger) and that with a positive control (without free radicals).

The tested mixture comprised HRP (3 μg/ml), scopoletine (5×10⁻⁶M), glutathione (10⁻⁴ to 5×10⁻⁵M), with or without peptide B1 (10⁻⁴ to 5×10⁻⁵ M). An oxygenated water (H₂O₂) solution was added immediately before reading the first fluorescence measure.

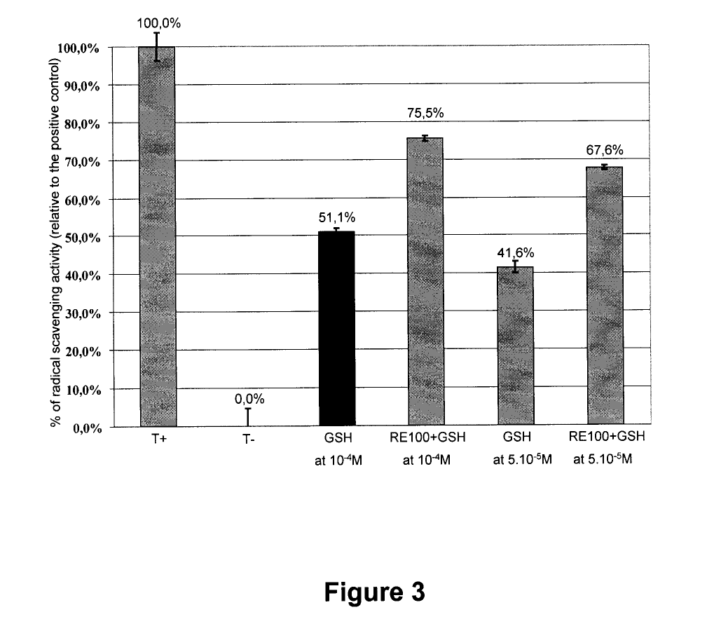

Results are presented in FIG. 3 and are expressed as the percentage of radical scavenging activity as compared to the positive control. Peptide B1 (RE100) displayed a glutathione regeneration activity that can be observed by an increase of glutathione scavenging activity as compared to in the absence thereof. T− is the negative control (without scavenger) and T+ is the positive control (without free radicals).

### Example 16

### Anti-Wrinkle Oil/Water Emulsion for Peptides of the Present Invention

| PHASES | % (w/w) |
|---|---|
| Oil Phase: | |
| Cetearyl alcohol (and) Cetearyl | 5 |
| glucoside (Montanov 68 ™) | |
| Jojoba oil | 5 |
| Vaseline oil | 5 |
| Isopropyl Palmitate | 7 |
| Aqueous Phase: | |
| Glycerine | 5 |
| Polyacrylamide and C13-14 | 0.3 |
| Isoparaffin and Laureth-7 (Sepigel | |
| 305 ™) | |
| Preservative agent | 0.5 |
| Perfume | 0.2 |
| Peptide A1 or B1 | 10 ppm |
| Water qsp | 100 |

### Example 17

Antiaging Cream Formulation for Peptides of the Present Invention

| CREAM | | % (w/w) |
|---|---|---|
| A | Polyoxyethylene (21) Stearyl | 2.00 |
| | Ether (Brij 721 ™) | |
| | Polyoxyethylene (2) Stearyl | 3.00 |
| | Ether (Brij 72 ™) | |
| | Stearyl alcohol (Hyfatol 18- | 4.00 |
| | 95 ™) | |
| | Caprylic/Capric Triglyceride | 4.00 |
| | (Myritol 318 ™) | |
| B | Hexyldecanol & Hexyldecyl | 5.00 |
| | Laurate (Cetiol PGL ™) | |
| | Polydimethylsiloxane polymer | 1.00 |
| | (Dow Corning 200 ™ fluid) | |
| C | Demineralized water | 27.40 |
| | Carbomer (Carbopol Ultrez | 0.10 |
| | 10 ™) | |
| | Demineralized water | 43.00 |
| | Peptide A1 or B1 | 10 ppm |
| | Panthenol (D-panthenol 75L ™) | 0.70 |
| D | D-Sorbito (Karion Liquid ™) | 3.00 |
| | Phenoxyethanol, | 0.50 |
| | methylparaben, butylparaben, | |
| | ethylparaben, propylparaben | |
| | (Phenonip ™) | |
| | Distarch Phosphate (Corn P04 | 6.00 |
| | Ph <<B>> ™) | |
| | NaOH 10% | 0.30 |
| | Perfume | |

### Example 18

### Sun Protection Formulation for Peptides of the Present Invention

| | % (w/w) |
|---|---|
| PHASE A: | |
| Xantham gum (Keltrol ™) | 0.40 |
| Glycerin 96% | 1.50 |
| Butylene Glycol | 1.00 |
| Tetrasodium EDTA | 0.10 |
| Caprylyl Glycol (lexgard o ™) | 1.00 |
| Peptide A1 or B1 | 10 ppm |
| Water | qsp |
| PHASE B: | |
| Octinoxate (Uvinul MC80 ™) | 7.50 |
| Oxybenzone (Uvinul M 40 ™) | 5.25 |
| Octisalate (Dermoblockos ™) | 5.00 |
| Homo Salate (Neo Heliopan ™ HMS) | 13.00 |
| Glyceryl Stearate/PEG stearate/ | 2.50 |
| (LEXEMUL 561 ™) | |
| Neopentyl glycol Diheptanoate | 2.25 |
| (LEXFEEL 7 ™) | |
| Adipic Acid/Diethylene glycol/ | 3.00 |
| Glycerine Crosspolymer | |
| (LEXOREZ 100 ™) | |
| PHASE C: | |
| Hydroxyethylacrylate/Sodium | 3.50 |
| Acryloydimethyl taurate copolymer, | |
| squalane and polysorbate 60 | |
| (SIMULGEL NS ™) | |
| PHASE D: | |
| Silica (MSS-500 ™) | 2.00 |

Although the present invention has been described hereinabove by way of specific embodiments thereof, it can be modified, without departing from the spirit and nature of the subject invention as defined in the appended claims.

1. A peptide of formula I (SEQ ID NO: 1):
Lip-A-Gly-His-B-R (I)
wherein:
Lip is a lipoyl residue of R or S configuration;
A is absent or is a lysine residue of configuration L or D;
Gly is a glycine residue;
B is a lysine residue of configuration L or D, or a lysine residue of configuration L or D in which the NH₂ group of the side chain comprises a modification, wherein said modification is (i) a replacement with a hydrogen or (ii) a modification with a protecting group selected from the group consisting of acetyl, benzoyl, tosyl, sulfonyl benzene, benzyloxycarbonyle and palmitoyl;
His is a histidine residue of configuration L or D; and
R is OZ or N(Z′)(Z″), wherein Z, Z′ and Z″ are independently of each other a hydrogen or a protecting group selected from the group consisting of methyl, ethyl, propyl, phenyl, hexyl, decyl and hexadecyl,
or a racemate, an enantiomer or a diastereomer thereof, or a mixture thereof, or a salt thereof.

2. The peptide of claim 1, wherein A is a lysine residue.

3. The peptide of claim 1, wherein B is a lysine residue of configuration L or D.

4. The peptide of claim 1, wherein R is NH₂.

5. The peptide of claim 1, wherein at least one of the lysine and histidine residues are in configuration L.

6. The peptide of claim 1, wherein the lysine and histidine residues are in configuration L.

7. The peptide of claim 1, wherein the lipoyl residue is in configuration R.

8. The peptide of claim 1, wherein the lipoyl residue is in configuration S.

9. The peptide of claim 1, wherein said peptide is Lip-Lys-Gly-His-Lys-NH₂ (SEQ ID NO: 3).

10. The peptide of claim 1, wherein said peptide is Lip-Lys-Gly-His-Lys (SEQ ID NO: 4).

11. The peptide of claim 1, wherein said peptide is Lip-Gly-His-Lys-NH₂ (SEQ ID NO: 5).

12. The peptide of claim 1, wherein said peptide is Lip-Gly-His-Lys (SEQ ID NO: 6).

13. The peptide of claim 1, wherein said peptide is Lip-Lys-Gly-His-Lys-N(CH₃)₂ (SEQ ID NO: 7).

14. The peptide of claim 1, wherein said peptide is Lip-Gly-His-Lys-N(CH₃)₂ (SEQ ID NO: 8).

15. A peptide of formula II (SEQ ID NO: 2)
Lip-A-His-B-C-Trp-R (II)
wherein:
Lip is a lipoyl residue of configuration R or S;
His is a histidine residue of configuration L;
Trp is a tryptophane residue of configuration L;
A is absent, is an amino acid residue of configuration L or D selected from the group consisting of a lysine residue, an alanine residue, a glutamic acid residue and a glycine residue, or is a spacer of formula: NH—(CH₂)n-CO— wherein n is an integer comprised between 2 and 14;
B is an aromatic amino acid residue of configuration D selected from the group consisting of a phenylalanine residue, a homophenylalanine residue, a tryptophane residue, a β-(1-Naphthyl)-alanine residue, a β-(2-Naphthyl)-alanine residue and a phenylglycine residue;
C is a basic amino acid residue of configuration L selected from the group consisting of an arginine residue, a lysine residue, an ornithine residue and a homoarginine residue; and
R is OZ or N(Z′)(Z″),
wherein Z, Z′ and Z″ are independently of each other a hydrogen or a protecting group selected from the group consisting of methyl, ethyl, propyl, phenyl, hexyl, decyl and hexadecyl,
or a racemate, an enantiomer or a diastereomer thereof, or a mixture thereof, or a salt thereof.

16. The peptide of claim 15, wherein the lipoyl residue is in configuration R.

17. The peptide of claim 15, wherein the lipoyl residue is in configuration S.

18. The peptide of claim 15, wherein A is absent.

19. The peptide of claim 15, wherein B is a phenylalanine residue.

20. The peptide of claim 15, wherein C is an arginine or an ornithine residue.

21. The peptide of claim 15, wherein C is an arginine residue.

22. The peptide of claim 15, wherein R is NH₂.

23. The peptide of claim 15, wherein said peptide is Lip-His-DPhe-Arg-Trp-NH₂ (SEQ ID NO: 9).

24. The peptide of claim 15, wherein said peptide is Lip-Lys-His-DPhe-Arg-Trp-NH₂ (SEQ ID NO: 10).

25. The peptide of claim 15, wherein said peptide is Lip-His-Trp-Arg-Trp-NH₂ (SEQ ID NO: 11).

26. The peptide of claim 15, wherein said peptide is Lip-Lys-His-Trp-Arg-Trp-NH₂ (SEQ ID NO: 12).

27. The peptide of claim 15, wherein said peptide is Lip-His-DPhe-Orn-Trp-NH₂ (SEQ ID NO: 13).

28. A composition comprising an effective amount of the peptide of claim 1, and a topically, cosmetically or pharmaceutically acceptable excipient or carrier.

29. The composition of claim 28, wherein said effective amount is between about 10⁻⁸M to about 10⁻²M.

30. The composition of claim 28, wherein said effective amount is between about 10⁻⁶M to about 10⁻⁵M.

31. The composition of claim 28, wherein said composition is a topical composition.

32. The composition of claim 31, wherein said composition is an aqueous solution, a cream, a water-in-oil emulsion, a oil-in-water emulsion, a gel, a spray, an ointment, a lotion, or a paste.

33. The composition of claim 28, further comprising at least one additional active agent.

34. The composition of claim 33, wherein said at least one additional active agent is a UV filter.

35. The composition of claim 33, wherein said at least one additional active agent is another peptide of Formula I or II.

36-57. (canceled)

58. A method of:
(i) preventing, reducing, delaying or treating a skin condition in a biological system; or
(ii) photo protecting a biological system against UVA- and/or UVB-induced damages; or
(iii) inhibiting IL-1α-induced IL-8 production in a biological system; or
(iv) increasing glutathione regeneration in a biological system; or
(v) increasing glutathione scavenging activity in a biological system; or
(vi) preventing, reducing, delaying or treating free radicals-induced inflammation in a biological system; or
(vii) any combination of (i) to (vi),
said method comprising administering an effective amount of the peptide of claim 1, to said biological system.

59. The method of claim 58, wherein said method is for preventing, reducing, delaying or treating a skin condition in a biological system, and said skin condition is an aging-related skin condition.

60. The method of claim 59, wherein said aging-related skin condition is the appearance or presence of (a) wrinkles, (b) fine lines or (c) both (a) and (b), on the skin.

61. The method of claim 58, wherein said method is for preventing, reducing, delaying or treating a skin condition in a biological system, and said skin condition is a skin injury.

62. The method of claim 61, wherein said skin injury is associated with surgical treatment, dermabrasion, laser treatment or peeling.

63. The method of claim 58, wherein said method is for preventing, reducing, delaying or treating a skin condition in a biological system, and said skin condition is a photo aging-related skin condition.

64. (canceled)

65. The method of claim 64, wherein said method is for photo protecting a biological system against UVA- and/or UVB-induced damages, and the damages include UV-induced DNA modifications.

66. The method of claim 64, wherein said method is for photo protecting a biological system against UVA- and/or UVB-induced damages, and the damages include UV-induced oxidative lesions.

67-70. (canceled)

71. The method of claim 58, wherein said biological system is a cell, a tissue or an organ.

72. The method of claim 71, wherein said cell is a skin cell.

73. The method of claim 71, wherein said organ is skin.

74. A kit or package comprising the peptide of claim 1, together with instructions for preventing, reducing, delaying or treating a skin condition in a subject.

75. The kit or package of claim 74, further comprising a UV filter.

76. A kit or package comprising the peptide of claim 1, and a container.

77-78. (canceled)
